(19) United States
(12) Patent Application Publication
(10) Pub. No.: US 2010/0028361 A1
(43) Pub. Date: Feb. 4, 2010

(54) BRAIN-DERIVED GONADOTROPINS AND COGNITION

(76) Inventors: Mark A. Smith, Chagrin Falls, OH (US); Kate M. Webber, Indianapolis, IN (US)

Correspondence Address: TAROLLI, SUNDHEIM, COVELL & TUMMINO, LLP, 1300 EAST NINTH STREET, SUITE 1700, CLEVELAND, OH, 44114, US

(21) Appl. No.: 12/520,320

(22) PCT Filed: Dec. 19, 2007
(86) PCT No.: PCT/US07/88057

§ 371 Date: Jun. 19, 2009

Related U.S. Application Data
(60) Provisional application No. 60/875,793, filed on Dec. 19, 2006.

(51) Int. Cl. A61K 39/395; A61K 31/7105; A61K 31/4164

(52) U.S. Cl. 424/158.1; 514/44 A; 514/397

(57) ABSTRACT

A method of treating or preventing neurodegenerative disease in a subject, the method includes administering to the subject a therapeutically effective amount of at least one physiologically acceptable agent that modulates levels, production, and/or function of brain-derived hormones of the hypothalamic-pituitary-gonadal (HPG) axis or their receptors.

### RELATED APPLICATION

This application claims priority from U.S. Provisional Application No. 60/875,793, filed Dec. 19, 2006, the subject matter, which is incorporated herein by reference.

### FIELD OF THE INVENTION

The present invention relates to method of treating neurodegenerative diseases, such as Alzheimer disease.

### BACKGROUND

Alzheimer disease (AD) is characterized by selective neuronal degeneration affecting the hippocampus as well as other cortical brain regions resulting in progressive memory loss and is the most prevalent neurodegenerative disease, affecting nearly 24 million people worldwide. Due to the extended course of AD, the etiologic events leading to the neuronal loss and dysfunction are difficult to determine, however, recent evidence supports a role for hormones, namely estrogen and testosterone, in AD pathogenesis. Although far from conclusive, epidemiological studies investigating gender differences in AD tend to support the higher prevalence and of AD in women. Since sex and age are two of the biggest risk factors for AD, it has logically been hypothesized that hormonal deficiency following reproductive senescence, which is more pronounced in females as is AD, may contribute to the etiology of AD.

### SUMMARY OF THE INVENTION

The present invention relates to a method of treating or preventing neurodegenerative disease in a subject. The method comprises administering to the subject a therapeutically effective amount of at least one physiologically acceptable agent that modulates levels, production, and/or function of brain-derived hormones or their receptors of the hypothalamic-pituitary-gonadal (HPG) axis. For example, the agent can reduce or eliminate levels, production, and/or function of brain-derived gonadotropin and/or gonadotropin receptor in the subject.

In an aspect of the invention, the agent can reduce or eliminate leutinizing hormone-s levels in the brain of the subject. The agent can be administered at an amount effective to also reduce or eliminate amyloid-β levels in the brain. The agent can also reduce the level of at least one of GnRH or GnRH receptor in the subject's brain.

In another aspect of the invention, the agent can comprise at least one of GnRH analogs, GnRH antagonists, GnRH receptor antagonists, anti-GnRH antibody, anti-GnRH receptor antibody, gonadotropin antagonists, gonadotropin receptor antagonists, anti-gonadotropin antibody, or anti-gonadotropin receptor antibody. One example of an agent that can be used in accordance with the invention is leuprolide or a physically acceptable analogs and salts thereof. Another example of an agent that can be used in accordance with the invention comprises interference RNA directed to mRNA that encodes gonadotropin, and/or gonadotropin receptor in the brain.

The present invention also relates to a method of treating or preventing Alzheimer disease (AD) in a subject. The method comprises administering to a subject a therapeutically effective amount of at least one physiologically acceptable agent that reduces or eliminates brain derived gonadotropins and/or brain derived gonadotropin receptors in the subject. The brain derived gonadotropin and/or gonadotropin receptor can comprise at least one of brain derived luteinizing hormone, brain derived luteinizing hormone receptor, brain derived human chorionic gonadotropin, and brain derived human chorionic gonadotropin receptor.

### BRIEF DESCRIPTION OF THE DRAWINGS

FIG. 1 is a graph illustrating cognitive performance as measured by a Y-maze. Percent number of alternations in a 5 minute trial for Tg-LHβ mice (Tg_LHβ) and Wild-type littermates (WT) (*=P<0.05).

FIG. 2 is a graph illustrating cognitive performance as measured by a Y-maze. Percent number of alternations in a 5 minute trial for LHRKO homozygous (−/−), heterozygous (+/−) and wild-type littermates (+/+) (*=P<0.05).

FIG. 3 is a plot illustrating Leuprolide, a gonadotropin-lowering drug, decreases brain Aβ levels in mice. C57B1/6J mice (3 months old) were administered either vehicle or a slow release leuprolide acetate (1.5 mg/kg; intraperitoneal monthly) mixture at 0 and 4 weeks. Mice were euthanized at 0, 4, and 8 weeks, the brains were dissected, the frontal cortex tissues were homogenized and centrifuged, and the supernatant analyzed for Aβ 1-40 and Aβ 1-42 levels via an Aβ ELISA assay. Results are expressed as picograms/mg of total protein (mean±S.D., n=6 mice at each time point). *, p<0.05; **, p<0.0001 for differences between vehicle and treated animals at the same time point.

FIG. 4 illustrates that LH induces Aβ secretion and insolubility in neuroblastoma cells. Human M17 neuroblastoma cells were cultured and treated with 0, 10, and 30 mIU/ml of LH for 5 days. Media with corresponding LH concentrations were replaced every 2 days. The medium from each experiment was used to measure secreted Aβ 1-40 (A). Cell pellets were solubilized in Triton X-100 and centrifuged to generate soluble (B) and insoluble fractions (C). Aβ concentration is expressed as picograms/mg of total protein (mean±S.D.). Experiments were performed three times in duplicate (i.e. n=6, p<0.01). LH receptor expression pattern in human M17 neuroblastoma cells was determined by immunoblot analysis with (D) a rabbit polyclonal antibody against residues 15-38 and (E) a mouse monoclonal antibody (3B5). Arrows indicate the immature (59 kDa) full-length LH receptor.

FIG. 5 is a graph illustrating Y-Maze performance in Tg2576 mice after leuprolide acetate (n=8) or saline treatment (n=5) at baseline and after 3 months. Figure illustrates the mean % alternations expressed as % change from baseline. (*) indicates significance at p<0.05.

FIG. 6 illustrates Aβ burden measured as % area stained in the entire hippocampus of 11 sections/brain/animal is significantly lower in animals treated with leuprolide acetate (n=8) compared to saline-treated animals (n=5, p<0.05). Representative image of Aβ burden in Tg2576 mice after saline (S) or leuprolide acetate (L). Scale bar, 200 μm.

FIG. 7 illustrates Leuprolide acetate treatment significantly reduces serum LH in Tg2576 mice (P<0.02). Inset: LHβ mRNA expression in mouse pituitary gland. Saline versus 6 and 8 week leuprolide acetate treatment.

FIG. 8 is a plot illustrating time course of serum LH following leuprolide acetate treatment.

FIG. 9 are graphs illustrating Y-maze performance after 3 months post-OVX or SHAM surgeries, Estrogen or placebo replaced (beginning at time of surgery) and treated with leuprolide acetate or saline. * Indicates a significant difference between saline and leuprolide acetate in the OVX+placebo group # indicates a significant difference between SHAM+saline and OVX+placebo+saline.

FIG. 10 are plots illustrating the length of time taken to find the invisible platform across three training days for OVX+estrogen (E) and placebo (P) replaced animals treated with leuprolide acetate (L) or saline (S) and sham operated animals (SHAM). (Significance indicated by: *=P+S vs E+S; #=P+S vs P+L; $=P+S vs SHAM; %=P+S vs E+L; at p<0.05).

FIG. 11 are plots illustrating the distance swam in NE quadrant (A); Latency to enter NE quadrant (B); Number of platform crossings (C); Latency to enter platform location (D), during the probe trial for OVX+ estrogen (E) and placebo (P) replaced animals treated with leuprolide acetate (L) or saline (S) and sham operated animals (SHAM).

FIG. 12 are graphs illustrating % Time spent in NE quadrant during the probe trial for OVX+ estrogen (E) and placebo (P) replaced animals treated with leuprolide acetate (L) or saline (S) and sham operated animals (SHAM).

FIG. 13 is a graph illustrating LH-β expression in AD v. control.

FIG. 14 is a graph illustrating GnHR expression in AD v. control.

FIG. 15 is a graph illustrating the effect of leuprolide acetate on brain-derived LH-β expression in C57B16/J female mice.

FIG. 16 is a graph illustrating the effect of leuprolide acetate on GnHR expression in C57B16/J female mice.

FIG. 17 illustrates an assay of HCG β mRNA expression in AD brain.

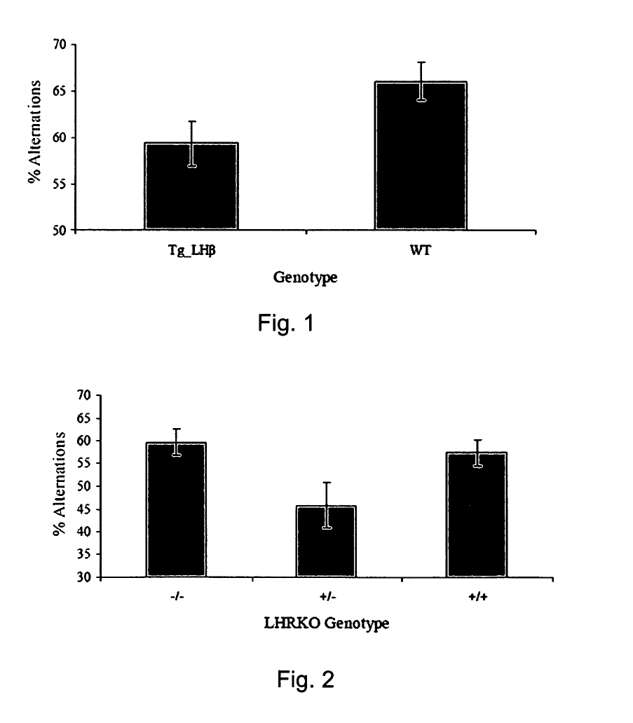

### DETAILED DESCRIPTION

The present invention relates to a method of treating or preventing neurodegenerative disease (e.g., AD disease) in a subject. The present invention is based on the discovery that luteinizing hormone-β (LH-β), human chorionic gonadotropin-β, and GnRH mRNA, but not FSH-β or α subunit mRNA, are found in Alzheimer disease and control hippocampal and cortical tissues and that there is a statistical increase of LH-β mRNA in Alzheimer disease versus age-matched control brains. Increased brain-derived gonadotropin levels (e.g., LH-β) of the subject in the presence of functional receptors may at least part be responsible for neurodegenerative diseases, such as Alzheimer disease. The examples of the present invention suggest that modulation of hormones of the hypothalamic-pituitary-gonadal (HPG) axis (e.g., LH-β) or their receptors levels can be used as a therapeutic strategy for neurodegenerative disease, such as Alzheimer disease.

According to this invention, modulating (e.g., decreasing) brain derived hormones of the hypothalamic-pituitary-gonadal (HPG) axis (e.g, LH-β or HCG) or their receptors in a subject can prevent, treat, and/or inhibit neurodegenerative diseases in the subject. Thus, the present invention entails a method of treating neurodegenerative disease, such as Alzheimer disease, in a person suffering therefrom and a method of preventing neurodegenerative disease in a person susceptible thereto by administration to the person a neurodegenerative disease treatment-effective amount or a neurodegenerative disease prevention-effective amount, respectively, of an agent, which will modulate hormones of the hypothalamic-pituitary-gonadal (HPG) axis (e.g, LH-β or HCG) or their receptors in the subject.

In an aspect of the invention, the agent can reduce the level of brain-derived gonadotropins or brain-derived gonadotropin receptors in the subject. Among such agents are those selected from the group consisting of GnRH analogs and physiologically acceptable salts thereof, GnRH antagonists, GnRH receptor antagonists, gonadotropin antagonists (e.g., LH antagonists, human chorionic gonadotropin (HCG) antagonists), gonadotropin receptor antagonists, vaccines that stimulate production of anti-GnRH antibodies, anti-GnRH receptor antibodies, anti-gonadotropin antibodies, or anti-gonadotropin receptor antibodies, or conjunctive administrations of such compounds.

A person or subject “suffering from AD” is a person who has been diagnosed as having AD, by a practitioner of at least ordinary skill in the art of clinically diagnosing AD, using methods and routines that are standard in the art of such clinical diagnoses.

By “treating AD” it is meant slowing or preventing the progression or worsening of the AD that is now known to occur when untreated.

By “preventing or treating” AD in a person susceptible thereto it is meant preventing the development of the disease in such a person to the point that the person would be clinically diagnosed, by a practitioner of at least ordinary skill in the art of diagnosing AD, as definitely suffering from AD.

In accordance with the invention, neurodegenerative disease in a subject can be treated by administration to the subject any composition that reduces the subject's brain-derived level of gonadotropin and/or gonadotropin receptor in an amount and for a duration effective to bring about such a reduction.

Further, in accordance with the invention, neurodegenerative disease (e.g., AD) in a subject can be prevented, or onset of clinical or behavioral manifestations delayed, in the subject by administration to the subject of any composition that reduces the level of a brain-derived gonadotropin or gonadotropin receptor in the subject in an amount and for a duration effective to bring about such a reduction to a level below, which development of neurodegenerative disease (e.g., AD) will not occur.

Reference herein to “level of a brain-derived gonadotropin and/or gonadotropin receptor” in a person or subject means the concentration of the biologically active gonadotropin and/or gonadotropin receptor in the subject's brain. Typically, the level of a brain-derived gonadotropin and/or gonadotropin receptor will be reduced by reducing the concentration of the brain-derived gonadotropin and/or gonadotropin receptor itself. However, reducing the activity of the brain-derived gonadotropin and/or gonadotropin, such as by binding it with an antibody that blocks the hormone's activity, even if the concentration of the brain-derived gonadotropin and/or gonadotropin receptor remains the same, is considered reducing the level of the brain-derived gonadotropin and/or gonadotropin receptor for purposes of the present application. The brain concentrations of gonadotropin and/or gonadotropin receptors in a human can be determined by any of a number of methods well known to the skilled.

As understood in the art, vaccines that stimulate production of antibodies can be employed to bind to brain-derived gonadotropins (e.g., LH-β and HCG-β), gonadotropin receptors, GnRH and/or GnRH receptors block or at least substantially reduce their biological activities. Thus, vaccine-stimulated antibodies to brain-derived gonadotropins (e.g., LH-β and HCG-β), gonadotropin receptors, GnRH and/or GnRH receptor can be employed in accordance with the invention to directly reduce the level of these proteins and thereby treat or prevent cognitive decline in post menopausal and post-hysterectomy subjects. Such antibodies to GnRH and/or GnRH receptor, by blocking its activity, will result in reduced levels of gonadotropins. These antibodies can be employed in accordance with the invention to reduce levels of gonadotropins and thereby to prevent or treat cognitive decline. Examples of such vaccines include the Talwar vaccine and a vaccine marketed under the tradename GONADIMMUNE by Aphton Corporation.

Antibodies for use in accordance with the invention may be made by conventional methods for preparation of vaccine antibodies for therapeutic use in humans. The vaccine-stimulated antibodies may be polyclonal and from any antibody-producing species, such as mice, rats, horses, dogs or humans. The antibodies may also be, and preferably are, monoclonal from cultures of antibody-producing cells from an antibody-producing species such as mice, rats, horses, dogs, and humans. The term “antibody” as used herein, unless otherwise limited, also encompasses antigen-binding fragments, such as F_{ab} fragments, of intact antibodies. If an antibody is monoclonal but from cultured cells of a species other than human, the antibody may be “humanized” by conventional methods to make it more tolerable immunologically to a person treated therewith. Antibodies for use in accordance with the invention can also be made by conventional techniques using cultured cells, preferably human cells, that have been genetically engineered to make a desired intact antibody or antigen-binding antibody fragment.

Antibodies will be administered in accordance with the invention by any method known in the art for administering same but preferably by intravenous injection of a sterile aqueous solution of the antibody, together with standard buffers, preservatives, excipients and the like.

GnRH analogs and pharmaceutically acceptable salts thereof can be employed to reduce levels of brain-derived gonadotropins (e.g., LH-β and HCG-β) to levels that are undetectable in the brain. Examples of GnRH analogs or salts thereof that may be employed in accordance with the invention include, for example, GnRH itself and its monoacetate and diacetate salt hydrates (Merck Index entry no. 5500) and the many analogs thereof that are known in the art. These include, for example, leuprolide and its monoacetate salt (Merck Index entry no. 5484, U.S. Pat. No. 4,005,063); the analogs of leuprolide with the D-leucyl residue replaced with D-aminobutyryl, D-isoleucyl, D-valyl or D-alanyl and the monoacetate salts thereof (U.S. Pat. No. 4,005,063); buserelin and its monoacetate salt (Merck Index entry no. 1527, U.S. Pat. No. 4,024,248); nafarelin and its monoacetate and acetate hydrate salts (Merck Index entry no. 6437, U.S. Pat. No. 4,234,571); deslorelin (Merck Index entry no. 2968); histrelin and its acetate salt (Merck Index entry no. 4760, U.S. Pat. No. 4,244,946); and goserelin and its acetate salt (Merck Index entry no. 4547, U.S. Pat. No. 4,100,274). For other GnRH analogs and salts thereof that can be used in accordance with the invention, see also U.S. Pat. No. 4,075,192, U.S. Pat. No. 4,762,717, and the U.S. patents cited at column 3, lines 49-54, of U.S. Pat. No. 4,762,717.

All of the U.S. patents cited herein, including those not cited specifically but cited at column 3, lines 49-54, of U.S. Pat. No. 4,762,717, and all of the Merck Index entries cited herein are incorporated herein by reference.

Administration of GnRH analogs in accordance with the invention will be by any method known in the art for administering same. Thus, administration may be by injection subcutaneously, intramuscularly or intravenously of a sterile aqueous solution which includes the analog together with buffers (e.g., sodium acetate, phosphate), preservatives (e.g., benzyl alcohol), salts (e.g., sodium chloride) and possibly various excipients or carriers. In this connection, see, for example, Physician's Desk Reference, 51. 8^{th} sup.st Ed., Medical Economics Co., Montvale, N.J., U.S.A. (1997), pp. 2736-2746 (leuprolide acetate) and pp. 2976-2980 (goserelin acetate), which are also incorporated herein by reference.

The dose and dosage regimen for a particular composition used to carry out the invention with a particular patient will vary depending on the active ingredient and its concentration and other components in the composition, the route of administration, the gender, age, weight, and general medical condition of the patient, and whether the patient is already suffering from cognitive decline. The skilled medical practitioner will be able to appropriately prescribe dosage regimens to carry out the invention. It is preferred in carrying out the invention that the concentrations of brain-derived gonadotropins (e.g., LH-β and HCG-β) and/or gonadotropin receptors be reduced to and maintained at levels that are as low as possible. It is usually preferred that the concentrations of brain-derived gonadotropins (e.g., LH-β and HCG-β) be reduced to undetectable levels.

In a another embodiment of carrying out the invention, a composition comprising a GnRH analog can be administered intramuscularly or subcutaneously as a depot composition from which release of the analog into the patient's system will be sustained over a long period, from about a week to about six months or more. This will maintain the concentration of gonadotropin in the brain of the subject at the low or undetectable level(s) as described above without the pain, cost and inconvenience of much more frequent (e.g., daily) administration. Such depot compositions of GnRH analogs are known and their preparation is well within the skill of the ordinarily person skilled in the art. See, e.g., Physician's Desk Reference, 51.8^{th} sup.st Ed. pp. 2736-2746 and 2976-2980, cited above.

Information from data already available or easily obtained by routine experimentation on GnRH analogs in suppressing gonadotropin activity, those of ordinary skill can easily determine the dose and dosage regimens for any GnRH analog.

Also useful in carrying out the invention are agents that antagonize the activity of GnRH. Agents that block the receptors for GnRH or to directly inhibit production of brain-derived gonadotropins or both, will result in reduced levels of brain-derived gonadotropins (e.g., LH-β and HCG-β) and can be employed in accordance with the invention to treat or prevent neurodegenerative disease, such as AD. Examples of GnRH antagonists include, for example, citrorelix and abberelix as well as GnRH antagonist disclosed in U.S. Patent Publication No. 2007/0191403, which is herein incorporated by reference in it entirety.

Other agents that can be used in the methods of the present invention include gonadotropin antagonists (e.g. Luteinizing hormone antagonists), gonadotropin receptor antagonists, and GnRH receptor antagonists as well as any agent or substance, which decreases the activity of brain-derived gonadotropins (e.g., LH-β and HCG-β) and/or gonadotropin receptors in the brain. The gonadotropin antagonists, gonadotropin receptor antagonists, GnRH antagonists, and GnRH receptor antagonists may physically bind to the brain-derived gonadotropins (e.g., LH-β and HCG-β) that facilitate neurodegenerative disease in the brain.

Examples of LH antagonists include milrinone, cilostamide, amrinone, enoximone, CI-930, anagrelide, pimobendan, siguazodan (SKF-94836), lixazinone (RS-82856), imazodan (CI-914), indolidan (LY195115), quazinone, SKF 94120, Org 30029, adibendan (BM 14,478), APP 201-533, carbazeran, cilostazole, E-1020, IPS-1251, nanterinone (UK-61260), pelrinone, RMI 82249, UD-CG 212, bemarinone (ORF-16,600) CK-2130, motapizone, OPC-3911, Ro 13-6438, sulmazole, vesnarinone (OPC-8212), buquineran, DPN 205-734, ICI-170777, isomazole (LY175326), MCI-154, MS-857, OPC-8490, piroximone (MLD 19205), RS-1893, saterinone, ZSY-39, and ICI 118233 as well as compounds disclosed in U.S. Pat. No. 6,297,243, which is herein incorporated by reference in its entirety.

In another aspect of the invention, the gonadotropin antagonists, gonadotropin receptor antagonists, GnRH antagonists, and GnRH receptor antagonists comprise RNA interference (RNAi) reagents to induce knockdown of brain-derived gonadotropins (e.g., LH-β and HCG-β), gonadotropin receptors, GnRH, and GnRH receptors or of a protein which transduces gonadotropin, gonadotropin receptor, GnRH, and GnRH receptor. RNAi is a process of sequence-specific post-transcriptional gene repression which can occur in eukaryotic cells. In general, this process involves degradation of an mRNA of a particular sequence induced by double-stranded RNA (dsRNA) that is homologous to that sequence. For example, the expression of a long dsRNA corresponding to the sequence of a particular single-stranded mRNA (ss mRNA) will labilize that message, thereby “interfering” with expression of the corresponding gene. Accordingly, any selected gene may be repressed by introducing a dsRNA which corresponds to all or a substantial part of the mRNA for that gene. It appears that when a long dsRNA is expressed, it is initially processed by a ribonuclease III into shorter dsRNA oligonucleotides of in some instances as few as 21 to 22 base pairs in length. Furthermore, RNAi may be effected by introduction or expression of relatively short homologous dsRNAs. Indeed the use of relatively short homologous dsRNAs may have certain advantages as discussed below.

Mammalian cells have at least two pathways that are affected by double-stranded RNA (dsRNA). In the RNAi (sequence-specific) pathway, the initiating dsRNA is first broken into short interfering (si) RNAs, as described above. The siRNAs have sense and antisense strands of about 21 nucleotides that form approximately 19 nucleotide si RNAs with overhangs of two nucleotides at each 3′ end. Short interfering RNAs are thought to provide the sequence information that allows a specific messenger RNA to be targeted for degradation. In contrast, the nonspecific pathway is triggered by dsRNA of any sequence, as long as it is at least about 30 base pairs in length. The nonspecific effects occur because dsRNA activates two enzymes: PKR, which in its active form phosphorylates the translation initiation factor eIF2 to shut down all protein synthesis, and 2′,5′ oligoadenylate synthetase (2′,5′-AS), which synthesizes a molecule that activates Rnase L, a nonspecific enzyme that targets all mRNAs. The nonspecific pathway may represents a host response to stress or viral infection, and, in general, the effects of the nonspecific pathway are preferably minimized under preferred methods of the present invention. Significantly, longer dsRNAs appear to be required to induce the nonspecific pathway and, accordingly, dsRNAs shorter than about 30 bases pairs are preferred to effect gene repression by RNAi (see Hunter et al. (1975) J Biol Chem 250: 409-17; Manche et al. (1992) Mol Cell Biol 12: 5239-48; Minks et al. (1979) J Biol Chem 254: 10180-3; and Elbashir et al. (2001) Nature 411: 494-8). In mammalian cells, siRNAs are effective at concentrations that are several orders of magnitude below the concentrations typically used in antisense experiments (Elbashir et al. (2001) Nature 411: 494-8).

The double stranded oligonucleotides used to effect RNAi are preferably less than 30 base pairs in length and, more preferably, comprise about 25, 24, 23, 22, 21, 20, 19, 18 or 17 base pairs of ribonucleic acid. Optionally the dsRNA oligonucleotides of the invention may include 3′ overhang ends. Exemplary 2-nucleotide 3′ overhangs may be composed of ribonucleotide residues of any type and may even be composed of 2′-deoxythymidine resides, which lowers the cost of RNA synthesis and may enhance nuclease resistance of siRNAs in the cell culture medium and within transfected cells (see Elbashi et al. (2001) Nature 411: 494-8). Longer dsRNAs of 50, 75, 100 or even 500 base pairs or more may also be utilized in certain embodiments of the invention. Exemplary concentrations of dsRNAs for effecting RNAi are about 0.05 nM, 0.1 nM, 0.5 nM, 1.0 nM, 1.5 nM, 25 nM or 100 nM, although other concentrations may be utilized depending upon the nature of the cells treated, the gene target and other factors readily discernable to the skilled artisan. Exemplary dsRNAs may be synthesized chemically or produced in vitro or in vivo using appropriate expression vectors. Exemplary synthetic RNAs include 21 nucleotide RNAs chemically synthesized using methods known in the art (e.g. Expedite RNA phosphoramidites and thymidine phosphoramidite (Proligo, Germany). Synthetic oligonucleotides are preferably deprotected and gel-purified using methods known in the art (see e.g. Elbashir et al. (2001) Genes Dev. 15: 188-200). Longer RNAs may be transcribed from promoters, such as T7 RNA polymerase promoters, known in the art. A single RNA target, placed in both possible orientations downstream of an in vitro promoter, will transcribe both strands of the target to create a dsRNA oligonucleotide of the desired target sequence.

If, for example, LH-β mRNA is the target of the double stranded RNA, any of the above RNA species will be designed to include a portion of a nucleic acid sequence that hybridizes, under stringent and/or physiological conditions to the LH-β mRNA. Likewise, if the target is the LH-β receptor mRNA, then any of the above RNA species will be designed to include a portion of a nucleic acid sequence that hybridizes, under stringent and/or physiological conditions to the corresponding mRNA sequence.

The specific sequence utilized in design of the oligonucleotides may be any contiguous sequence of nucleotides contained within the expressed gene message of the target. Programs and algorithms, known in the art, may be used to select appropriate target sequences. In addition, optimal sequences may be selected utilizing programs designed to predict the secondary structure of a specified single stranded nucleic acid sequence and allowing selection of those sequences likely to occur in exposed single stranded regions of a folded mRNA. Methods and compositions for designing appropriate oligonucleotides may be found, for example, in U.S. Pat. No. 6,251,588, the contents of which are incorporated herein by reference. Messenger RNA (mRNA) is generally thought of as a linear molecule which contains the information for directing protein synthesis within the sequence of ribonucleotides, however studies have revealed a number of secondary and tertiary structures that exist in most mRNAs. Secondary structure elements in RNA are formed largely by Watson-Crick type interactions between different regions of the same RNA molecule. Important secondary structural elements include intramolecular double stranded regions, hairpin loops, bulges in duplex RNA and internal loops. Tertiary structural elements are formed when secondary structural elements come in contact with each other or with single stranded regions to produce a more complex three-dimensional structure. A number of researchers have measured the binding energies of a large number of RNA duplex structures and have derived a set of rules which can be used to predict the secondary structure of RNA (see e.g. Jaeger et al. (1989) Proc. Natl. Acad. Sci. USA 86:7706 (1989); and Turner et al. (1988) Annu. Rev. Biophys. Chem. 17:167). The rules are useful in identification of RNA structural elements and, in particular, for identifying single stranded RNA regions which may represent preferred segments of the mRNA to target for silencing RNAi, ribozyme or antisense technologies. Accordingly, preferred segments of the mRNA target can be identified for design of the RNAi mediating dsRNA oligonucleotides as well as for design of appropriate ribozyme and hammerheadribozyme compositions of the invention.

The dsRNA oligonucleotides may be introduced into the cell by transfection with an heterologous target gene using carrier compositions such as liposomes, which are known in the art—e.g. Lipofectamine 2000 (Life Technologies) as described by the manufacturer for adherent cell lines. Transfection of dsRNA oligonucleotides for targeting endogenous genes may be carried out using Oligofectamine (Life Technologies). Further compositions, methods and applications of RNAi technology are provided in U.S. Pat. Nos. 6,278,039, 5,723,750 and 5,244,805, which are incorporated herein by reference.

Ribozyme molecules can be designed to catalytically cleave encoding mRNAs, or mRNAs encoding other proteins involved in gonadotropin activity and signalling (e.g., gonadotropin receptors, GnRH, and GnRH receptor). Ribozymes are enzymatic RNA molecules capable of catalyzing the specific cleavage of RNA. (For a review, see Rossi (1994) Current Biology 4: 469-471). The mechanism of ribozyme action involves sequence specific hybridization of the ribozyme molecule to complementary target RNA, followed by an endonucleolytic cleavage event.

While ribozymes that cleave mRNA at site specific recognition sequences can be used to destroy target mRNAs, the use of hammerhead ribozymes is preferred. Hammerhead ribozymes cleave mRNAs at locations dictated by flanking regions that form complementary base pairs with the target mRNA. The construction and production of hammerhead ribozymes is well known in the art and is described more fully in Haseloff and Gerlach (1988) Nature 334:585-591; and see PCT Appln. No. WO89/05852, the contents of which are incorporated herein by reference). Hammerhead ribozyme sequences can be embedded in a stable RNA such as a transfer RNA (tRNA) to increase cleavage efficiency in vivo (Perriman et al. (1995) Proc. Natl. Acad. Sci. USA, 92: 6175-79; de Feyter, and Gaudron, Methods in Molecular Biology, Vol. 74, Chapter 43, “Expressing Ribozymes in Plants”, Edited by Turner, P. C, Humana Press Inc., Totowa, N.J.). In particular, RNA polymerase III-mediated expression of tRNA fusion ribozymes are well known in the art (see Kawasaki et al. (1998) Nature 393: 284-9; Kuwabara et al. (1998) Nature Biotechnol. 16: 961-5; and Kuwabara et al. (1998) Mol. Cell 2: 617-27; Koseki et al. (1999) J Virol 73: 1868-77; Kuwabara et al. (1999) Proc Natl Acad Sci USA 96: 1886-91; Tanabe et al. (2000) Nature 406: 473-4). There are typically a number of potential hammerhead ribozyme cleavage sites within a given target cDNA sequence. Preferably the ribozyme is engineered so that the cleavage recognition site is located near the 5′ end of the target mRNA—to increase efficiency and minimize the intracellular accumulation of non-functional mRNA transcripts. Furthermore, the use of any cleavage recognition site located in the target sequence encoding different portions of the C-terminal amino acid domains of, for example, long and short forms of target would allow the selective targeting of one or the other form of the target, and thus, have a selective effect on one form of the target gene product.

Gene targeting ribozymes necessarily contain a hybridizing region complementary to two regions, each of at least 5 and preferably each 6, 7, 8, 9, 10, 11, 12, 13, 14, 15, 16, 17, 18, 19 or 20 contiguous nucleotides in length of a target mRNA. In addition, ribozymes possess highly specific endoribonuclease activity, which autocatalytically cleaves the target sense mRNA. The present invention extends to ribozymes which hybridize to a sense mRNA encoding a brain-derived gonadotropin (e.g., LH-β and HCG-β), gonadotropin receptor, GnRH, and GnRH receptor.

Ribozymes can be composed of modified oligonucleotides (e.g., for improved stability, targeting, etc.) and should be delivered to cells which express the target gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous target messages and inhibit translation. Because ribozymes, unlike antisense molecules, are catalytic, a lower intracellular concentration is required for efficiency.

In certain embodiments, a ribozyme may be designed by first identifying a sequence portion sufficient to cause effective knockdown by RNAi. The same sequence portion may then be incorporated into a ribozyme.

In certain embodiments, expression of the “target gene, whether it is a brain-derived gonadotropin (e.g., LH-β and HCG-β), gonadotropin receptor, GnRH, and GnRH receptor gene may be inhibited by an inhibitor RNA that is a single-stranded RNA molecule containing an inverted repeat region that causes the RNA to self-hybridize, forming a hairpin structure (a so-called “hairpin RNA” or “shRNA”). shRNA molecules of this type may be encoded in RNA or DNA vectors. The term “encoded” is used to indicate that the vector, when acted upon by an appropriate enzyme, such as an RNA polymerase, will give rise to the desired shRNA molecules (although additional processing enzymes may also be involved in producing the encoded shRNA molecules). The expression of shRNAs may be constitutive or regulated in a desired manner.

A double-stranded structure of an shRNA is formed by a single self-complementary RNA strand. RNA duplex formation may be initiated either inside or outside the cell. Inhibition is sequence-specific in that nucleotide sequences corresponding to the duplex region of the RNA are targeted for genetic inhibition. shRNA constructs containing a nucleotide sequence identical to a portion, of either coding or non-coding sequence, of the target gene are preferred for inhibition. RNA sequences with insertions, deletions, and single point mutations relative to the target sequence have also been found to be effective for inhibition. Because 100% sequence identity between the RNA and the target gene is not required to practice the present invention, the invention has the advantage of being able to tolerate sequence variations that might be expected due to genetic mutation, strain polymorphism, or evolutionary divergence. Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript. In certain preferred embodiments, the length of the duplex-forming portion of a shRNA is at least 20, 21 or 22 nucleotides in length, e.g., corresponding in size to RNA products produced by Dicer-dependent cleavage. In certain embodiments, the shRNA construct is at least 25, 50, 100, 200, 300 or 400 bases in length. In certain embodiments, the shRNA construct is 400-800 bases in length. shRNA constructs are highly tolerant of variation in loop sequence and loop size. An endogenous RNA polymerase of the cell may mediate transcription of an shRNA encoded in a nucleic acid construct. The shRNA construct may also be synthesized by a bacteriophage RNA polymerase (e.g., T3, T7, SP6) that is expressed in the cell.

The foregoing description, discussion and scope of the invention are directed to those of ordinary skill in the treatment of actual or incipient AD. Accordingly, it is to be expected that the teachings herein will enable selection of specific agents and regimens for treatment within the scope of the appended claims.

### EXAMPLES

### Example 1

### Increases in Luteinizing Hormone are Associated with Declines in Cognitive Performance

In this study, we herein evaluated cognitive performance in two transgenic mouse strains, both with high LH but only one with functional LH receptors. LH receptors, as mentioned before, are found in high levels in the hippocampus, a region critical in the pathogenesis of AD. Therefore, testing LH-over-expressor mouse model such as the Tg-LHβ in addition to the LHRKO mouse models on a hippocampally-dependent task may allow us to determine whether cognitive changes are modulated as well as whether the changes are receptor specific in this region.

Additionally, an added bonus of these models is that Tg-LHβ and LHRKO have differential estrogen status. That is, while Tg-LHβ mice show high LH levels and high estrogen levels, LHKO mice show high LH levels but none-functional receptors and therefore below average levels of estrogen. This is important and relevant to AD since, as mentioned above, estrogen (declines) has been associated with AD/age-related cognitive declines.

### Methods

### Development and Characterization of Transgenic Lines Tg-LHβ Mice:

Transgenic mice expressing a chimeric LH β subunit (LHβ) containing the C-terminal peptide of the human chorionic gonadotropin β subunit under the control of the αGSU promoter were previously described. All mice originated from one founder line and were F1 hybrids of CF-1 and FVB strains. Targeted expression of the LHβ chimera leads to elevated LH levels and infertility in female transgenic animals as well as increased estradiol and testosterone levels when compared to non-transgenic littermates.

### LH/hCG Receptor Gene Knockout Mice

LH/hCG receptors were disrupted by gene targeting in embryonic stem cells. The disruption resulted in infertility in both sexes and gonads and nongonadal tissues contained no receptor mRNA or receptor protein. The generation of this mouse is described in detail elsewhere. Briefly, a single gene with multiple transcription initiation sites present in the 5′-flanking region that encodes multiple transcripts and usually a single LH receptor protein was completely inactivated in the body using a targeting vector that deleted a part of the 5′-flanking region containing the promoter region and multiple transcription initiation sites, as well as most of exon 1. Disruption of the LH receptor gene led to increased levels of LH, decreased levels of estradiol and progesterone, and non-detectable levels of testosterone.

### Animals and Housing

For cognitive assessment (see below) we analyzed 9 transgenic LH-overexpressing female mice (Tg-LHβ) with an average age of 10 months and 15 age-matched non-transgenic littermates (average age 10 months). Additionally, we used 12 homozygous, 13 heterozygous transgenic LH receptor knock-out mice (LHRKO) and 8 age-matched wild-type littermates (average age 8 months). All animals were group housed, provided ad libitum access to food and water, and maintained on a 12 hr light/dark cycle. The Institutional Animal Care and Use Committee of Case Western Reserve University approved all animal studies.

### Behavioral Assessment—Y-Maze

To measure spontaneous alternation behavior and exploratory activity, a hippocampal-associated task, we used a Y-maze [32 cm (long)×10 cm (wide) with 26-cm walls]. Tg-LHβ and LHRKO animals were tested as previously described. Briefly, each animal (randomized and investigator-blinded) was placed in one of three arms of the Y-maze (alternating arms across animals in each group) and each arm entry was recorded for duration of 5 minutes. An alternation was defined as 3 entries in 3 different arms (i.e., 1, 2, 3 or 2, 3, 1 etc). % number of alternations was calculated as (total alternations/total number of entries−2)×100. The maze was cleaned with ethanol between each animal to minimize odor cues.

### Statistical Analysis

A Student's T-test comparing the Y-maze performance in the Tg-LHβ mice versus aged-matched controls was used to determine statistical significance with assistance of statistical analysis software Sigmastat (SPSS, Inc., Chicago, Ill.). Statistical significance was determined at the p<0.05 level. A one-way analysis of variance (ANOVA) was used to determine Y-maze performance differences between homozygous (−/−), heterozygous (+/−) and wild-type (+/+) LHRKO mice. Multiple comparisons using the Fisher LSD test were carried out to determine statistically significant differences across each individual group.

### Results

Tg-LHβ mice demonstrated significant declines in Y-maze performance when compared to non-transgenic littermates (t_{1,21}=−6.712, p<0.05) in the absence of differences in overall exploratory activity (t_{1,21}=−1.626, p=0.119). In mice that harbored a disrupted LH receptor (LHRKO), there were no significant differences between homozygous and wild-type mice (t_{1,24}=0.316, p=1.0), however a statistically significant group effect was present (F_{2, 31}=4.846, p<0.05) illustrating that heterozygous mice performed significantly worse than homozygous mice (t_{1,24}=2.923, p<0.05). As with Tg-LHβ, there were no differences in overall exploratory activity in the LHRKO mice across groups (F_{2,31}=0.895, p=0.419). To exclude possible sex or age-driven confounds, preliminary analyses prior to grouping animals across different gender and ages revealed no significant differences in Y-maze performance therefore data were collapsed across these variables for all subsequent analyses.

### Discussion

In this study we demonstrate that Tg-LHβ animals show declines in hippocampally-associated cognitive performance as measured by the Y-maze task. Previous reports reveal that LH is capable of modulating cognitive behavior and a more recent study demonstrates that experimental ablation of LH by a selective GnRH agonist (leuprolide acetate) improves Y-maze performance and decreases amyloid-β load in the hippocampus of APP transgenic mice. Given that LH is capable of crossing the blood-brain-barrier and that the highest level of LH receptors in the brain are localized to the hippocampus, an area that is highly vulnerable to both aging and AD, our data suggest that declines in hippocampally-related function may be associated with chronic LH elevation as seen in these mice or during menopause and AD in humans. With this in mind, the fact that Tg-LHβ animals sustain such high elevations in LH in addition to other elevated hormones (prolactin, corticosteroids, progesterone and testosterone) raises the possibility that the behavioral declines observed in these animals were mediated by indirect mechanisms rather than specifically via the LH receptor. To begin to address this issue, we also measured Y-maze performance in mice lacking functional LH receptors and found that homozygous knockout mice were indistinguishable from wild-type mice despite the fact that homozygous knockout mice show high elevations in LH; importantly, in the presence of reduced estrogen levels. On the other hand, our data also indicated that heterozygous knockout mice performed significantly worse than the two other groups in this task. While these results are somewhat surprising given that heterozygous mice are indistinguishable from wild-types in terms of hormonal levels (LH and estradiol), one possibility, which we are currently investigating, is that lower numbers of receptor under equal amounts of ligand leads to a potentiation response with receptors firing twice as much as they would normally do, thereby mimicking the findings observed for the Tg-LHβ animals.

Noteworthy, neither Tg-LHβ nor LHRKO animals showed declines in spontaneous alternation behavior in the absence of differences in overall exploratory activity. This supports the notion that the declines in Y-maze performance are hippocampal-specific rather than associated with a more general phenomenon such overall poorer health or tumor development in these animals. Furthermore, changes in estrogen levels in these animals were unlikely to be responsible for the cognitive changes observed in this study since Tg-LHβ mice show elevated, rather than diminished, levels of estrogen; LHRKO homozygous mice show decreased levels; and heterozygous knockout animals show equivalent levels of estrogen when compared to wild-type littermate controls. Therefore, and perhaps mimicking the situation in elderly-post-menopausal women undergoing HRT, estrogen levels appear not to be directly linked to declines in cognitive performance unless one takes into account the interrelationship with LH levels and LH receptor integrity. Notably, such an interrelation would explain the puzzling results described in the literature regarding the effectiveness of HRT to prevent cognitive decline and AD in post-menopausal women. Specifically, we suspect that increased dementia after HRT in elderly women (age 65 and above) may be attributable to the fact that while levels of estrogen were returned to pre-menopausal levels, levels of LH remain elevated and do not return to normal since the HPG axis feedback loop system, after years of chronic low estrogen and high gonadotropin levels, has already shut down. On the other hand, if HRT is started during peri- or early menopause, when the HPG axis feedback loop system is still functional, replacement of estrogen leads to the lowering of LH, and from epidemiological evidences, offers protection from age-related cognitive decline and AD.

In conclusion, our findings suggest that when examining declines in cognitive performance after menopause or during AD we should be careful to examine all the players involved in the equation rather than focusing on a single hormone. By solely focusing on estrogen we may be overlooking an ignored but very important partner, namely LH. In this regard, studies are currently underway to dissect the role of estrogen from that of LH using an ovariectomy as a model of menopause. More importantly, establishing the mechanism behind LH-related cognitive declines and targeting the release of LH may indeed be a successful strategy to prevent and forestall the progression of AD, illustrated by pre-clinical data using leuprolide acetate (Bowen et al., 2004; Casadesus et al., 2006) and a recently completed phase II clinical trial showing stabilization in cognitive impairment and activities of daily living in AD patients treated with high doses of leuprolide acetate. These promising findings support the importance of LH in AD and give way for an alternative and much needed therapeutic avenue for this insidious disease.

### Example 2

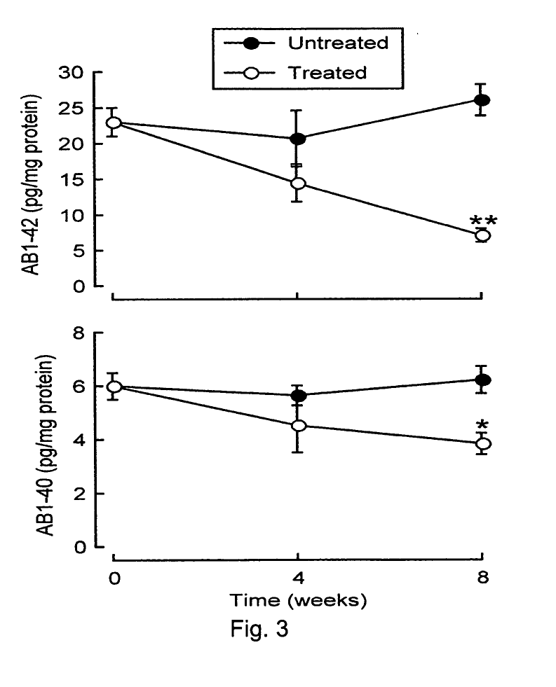

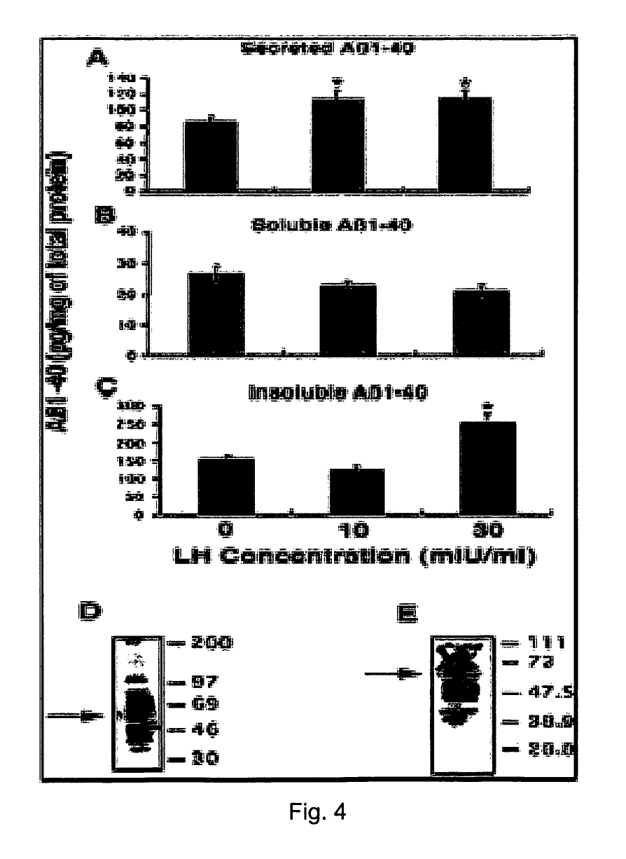

Based on these preliminary studies indicating that LH could be a factor in cognition and given that our group has previously reported that LH modulates amyloidogenic processing of AβPP (FIGS. 3 and 4), we evaluated the therapeutic potential of LH ablation, using a gonadotropin-releasing hormone analogue, leuprolide acetate, in aged (21 month old) Tg2576 female mice.

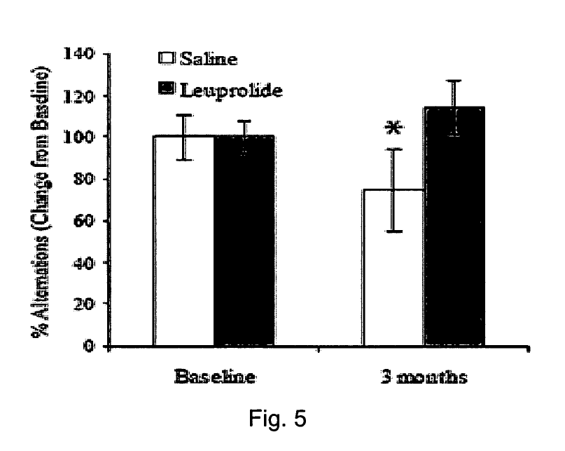

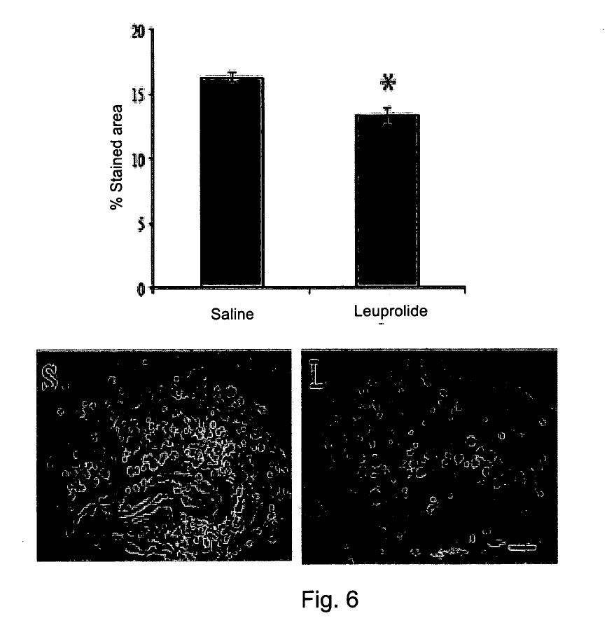

In this initial study we used aged animals to 1) circumvent the estrogen issue, since aged mice, like humans show estropause and 2) to examine the effects of LH ablation once the disease is well established. Our data indicates that LH ablation significantly attenuates cognitive decline (FIG. 5) (p<0.01) and decreases Aβ plaque load (p<0.05) (FIG. 6) as compared to placebo-treated animals. Therefore, our data suggests that, at least in aged AβPP transgenic mice, the positive effects of LH ablation override any negative effects of estrogen depletion. Importantly, while alternation behavior also depends on the innate tendency/preference of the animal to alternate, leading to the possibility that treatment, rather than improving/sustaining memory, could increase alternating preference, the fact that our data shows sustained rather than improved behavioral output in the treated animals compared to controls and the fact that treated animals did not show increases in overall arm entries nor any directional biases suggests that treatment did indeed sustain short-term memory rather than potentiate their preference to alternate.

Importantly, leuprolide acetate-mediated reductions of Aβ (p<0.05) was negatively correlated with improved cognition (r=−0.75, p<0.05). Such an assertion is in concert with data demonstrating that the modulation of estrogen in the AβPP/PS-1 animal model of AD leads to improvements in cognitive behavior but, and unlike our findings, no changes in pathological features of AD. This discrepancy in results could be explained by a differential LH status in the animals of the two studies since while in our study we ablated both estrogen and LH concurrently, OVX leads to declines in estrogen but a rise in the levels of LH and administration of estrogen (c.f., HRT) does not decrease LH levels beyond baseline. Therefore, one possibility is that it is only the decrease in estrogen when it is coupled with an increase in LH that leads to behavioral impairments and it is only the ablation of LH that leads to changes in Aβ pathology in these mice.

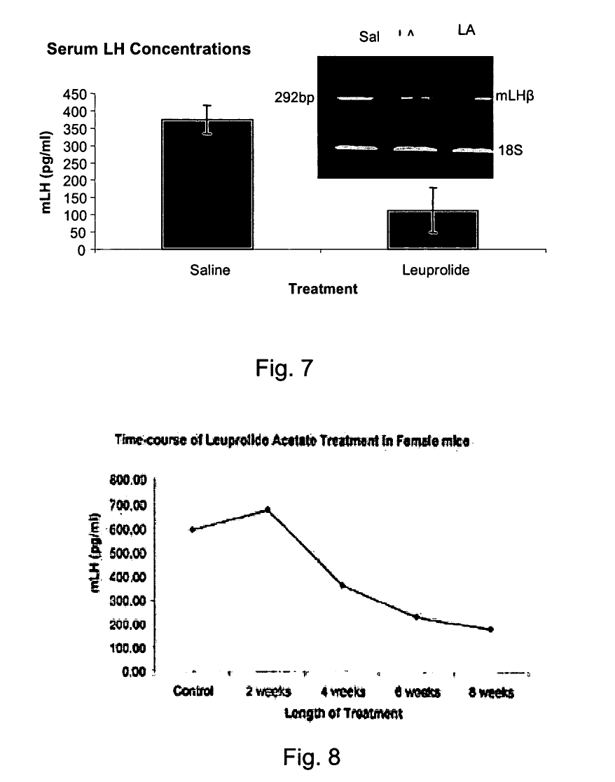

Treatment with leuprolide acetate causes a significant decline in serum LH (FIG. 7) paralleling a clear decrease in the expression of LHβ mRNA in the pituitary (FIG. 7 inset) in the mice used for this proposal. Similarly, a time-course (up to 8 weeks) following leuprolide acetate treatment depicts the classical LH modulation pattern by GnRH agonists (FIG. 8). These data support the notion that treatment with leuprolide acetate in mice follows the same pattern as treatment in human patients with regards to serum LH level modulation.

### Example 3

We present preliminary data gathered from a pilot study and that focuses on the effects of leuprolide acetate on cognition in ovariectomized animals with or without estrogen replacement immediately after surgery.

For this pilot 2 month old C57/B6 female mice from Jackson Labs, were ovariectomized (n=40) them and either replaced them with 90-day time-release estrogen (n=20) or placebo pellets (Innovative research of America, Fl) (n=18). 24 hours after surgeries and pellet implantations half of the animals were treated with either 0.9% saline an the other half with leuprolide acetate (7.5 mg/kg) for 3 months in an identical fashion to that previously reported (Casadesus et al., 2006). In addition we added a SHAM ovariectomized group that received saline and a placebo pallet (n=10). During the last two weeks of the study all animals have been tested for Y-maze and Morris Water Maze performance.

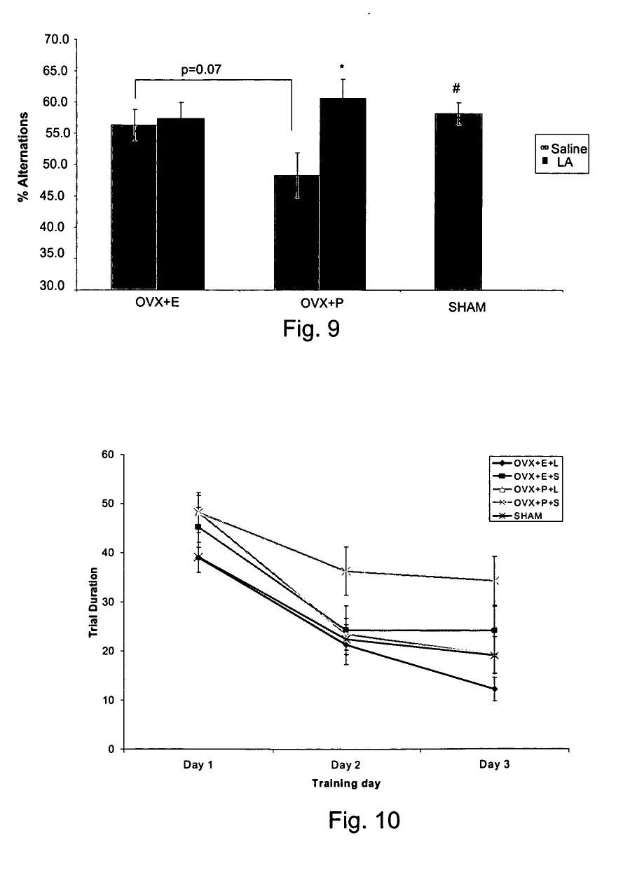

This supplemental material using Y-maze as a broad measure of cognitive function supports the tenants of our proposal as we have found that leuprolide treatment in OVX placebo-implanted mice was effective at improving OVX-dependent cognitive declines. Specifically, we found a group difference of drug treatment (leuprolide vs saline) when comparing OVXed animals that were replaced with estrogen or placebo (F=5.145; p=0.02). That is, leuprolide acetate significantly improved Y-maze performance as compared to saline-treated animals. Post-hoc analyses demonstrated that that this significant improvement of cognitive output was specific to the placebo replaced group (t=2.939; p=0.005). Additionally, our data indicates a strong trend (t=1.841; p=0.07) towards significance when comparing saline-treated estrogen-replaced animals versus saline-treated placebo replaced OVX animals (FIG. 9).

We feel that lack of statistical significance in the surgical parameter was likely due to the relatively small n number in each group rather than lack of success of the surgery protocol for two reasons: 1) that we observed a significant decline in cognitive performance in the OVX+Placebo+Saline group compared to the SHAM+sal group (F=5.802; p=0.02) and 2) that we observed a significant increase in body weight in the OVX+placebo group as compared to OVX+estrogen group (F=5.649; p<0.001) irrespective of drug treatment. Importantly, the OVX+estrogen replaced group was not different from the SHAM operated group, hence indicating that indeed estrogen replacement had an effect. We will be able to further confirm this point when we sacrifice the animals and send out blood samples for estrogen and gonadotropin measurements.

TABLE 1
Body weight table after 3 months post-
surgery + replacelement + drug treatment

| | | |
|---|---|---|
| OVX + | OVX + | |
| Estrogen | Placebo | SHAM |

| LA | Saline | LA | Saline | Saline |
|---|---|---|---|---|
| 22.1 | 23.5 | 26.3 | 28.5 | 22.8 |
| 0.2 | 0.4 | 1.6 | 1.3 | 0.4 |

As mentioned before, all mice also underwent MWM testing, a measurement of hippocampal function based on the capacity of the animal to find a hidden platform under water by remembering and using spatial cues in the environment. Importantly, these data further support our findings in the Y-maze. To this end, here we demonstrate that OVX in our protocol was successful at producing cognitive decline in our mice (F=5.308; p=0.03) when compared SHAM operated animals and that this cognitive decline was rescued by estrogen replacement (F=4.073; p=0.05), as measured by the length of time taken to locate the invisible platform across days (FIG. 10). More importantly, our data also indicates that leuprolide acetate was as effective as estrogen in rescuing OVX-associated cognition decline (F=5.176; p=0.028) and that overall, independent of replacement regiment, animals treated with leuprolide acetate learned at a faster rate than did animals treated with saline (F=3.783; p=0.027). Importantly, as indicated below (FIG. 10) all groups showed a progressive decline in time spent to find the hidden platform (F=82.378; p<0.001), thus Confirming that indeed the training worked and the OVX+placebo treated with saline performed significantly more poorly compared to the rest of the groups on days 2 and 3.

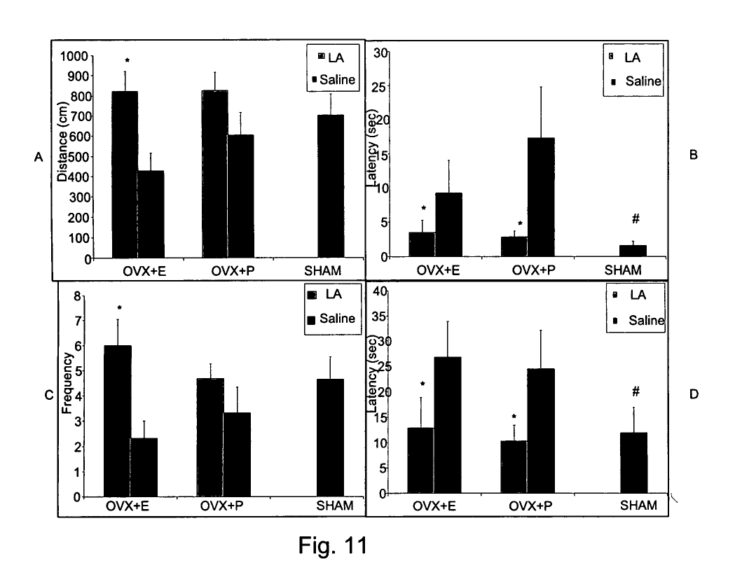

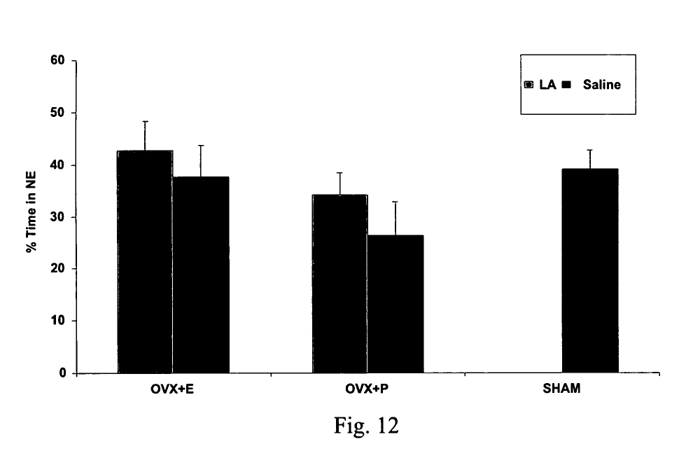

Additionally, to further determine the memory function and training success in the animals we measured the capacity of the animals to retain the information learned using a probe trial. In this regard, at the end of the last day of training we removed the platform and let the animals swim for 1 minute. We found that leuprolide treated animals swam longer distances in the quadrant that had previously held the platform (NE quadrant) (F=9.655; p=0.003) entered that quadrant earlier (F=4.427;0.041), crossed the invisible platform region more earlier (5.028; p=0.005) and crossed it more times (F=9.115; p=0.004) regardless of replacement regiment (FIG. 11) and that, there was a strong trend of replacement regiment towards significance for % time spent in the NE trial (FIG. 12).

We feel that this data strongly further supports our hypothesis as evidenced by the fact that ablation of gonadotropins has a significant positive impact on cognition in ovariectomized/estropausal animals. This data supports our findings in previous published literature using an AD transgenic model.

### Example 4

### Brain-Derived Gonadotropins and Cognition

While most research evaluating how differences in gender relate to the disease is primarily focused on the sex steroids, estrogen and testosterone, there a number of other hormones involved in the hypothalamic-pituitary-gonadal (HPG) axis axis that, along with estrogen and testosterone, regulate reproductive function. Among these hormones are the gonadotropins: luteinizing hormone (LH), human chorionic gonadotropin (hCG), follicle-stimulating hormone (FSH), and thyroid stimulation hormone (TSH). Interestingly, receptors for these other hormones are expressed in many non-reproductive tissues including, most notably, the brain. Considering this fact and the reported incomplete protection of hormone replacement therapy (HRT), we hypothesize that gonadotropic hormones, may be playing a central role in the pathogenesis of AD.

Gonadotropins are hormones of the HPG axis that control the synthesis and secretion of the sex steroids, and they were initially implicated in AD pathogenesis beginning with the finding of a two-fold increase in circulating gonadotropins in individuals with AD compared with age-matched control individuals. LH, in particular, has also been shown to alter amyloid β precursor protein (AβPP) processing toward the amyloidogenic pathway as evidenced by increased secretion and insolubility of Aβ, decreased AβPP-α secretion, and increased AβPP-C99 levels. These potentially pathogenic, LH-induced modifications of AβPP processing may in part be responsible for the cognitive decline seen in LH-β transgenic mice that exhibit elevated LH levels well as increased estradiol and testosterone levels when compared to non-transgenic littermates.

Importantly, significant elevations of LH were not only found in the serum of AD patients, but LH was also significantly increased in pyramidal neurons in the hippocampus of AD patients when compared to normal individuals. Because LH is thought to be produced solely by gonadotrophs in the pituitary, increased neuronal LH in the hippocampus of AD patients may be the result of increased serum LH crossing the blood-brain barrier as serum LH is also increased in AD. While the sex steroids are known to cross the blood-brain barrier due to their hydrophobic nature, less is known about the ability of gonadotropins, which are peptide hormones and therefore hydrophilic, to cross the blood-brain barrier. hCG, a gonadotropin that is highly homologous to LH, has the ability to cross the blood-brain barrier albeit not freely, however, similar studies have not been preformed with LH. Furthermore, if LH is able to cross the blood-brain barrier, it is debatable that this diffuse flow of LH into the brain could account for the increased neuronal LH in AD as the hypothetical mechanism by which serum LH is sequestered to neuronal cytoplasm is far from being determined.

Because it is ability of LH to not only cross the blood-brain barrier, but also to be endocytosed by neurons has yet to be demonstrated, we hypothesize that increased neuronal LH in AD may instead be the consequence of increased endogenous, brain-derived LH expression. While the ability of the brain to synthesize gonadotropins such as LH has yet to be studied aside from the report herein, it has been well documented that neurons in various regions of the central nervous system synthesize sex that are believed to be important for complex neuronal functions including hippocampal synaptic plasticity. Since serum gonadotropins are intimately linked to sex steroid synthesis and secretion, and along with gonadotropin-releasing hormone (GnRH) comprise the HPG axis, the presence of neurosteroids in the brain suggests that the brain may also be capable of expressing gonadotropins and GnRH forming an “HPG-like” axis contained within the brain itself.

In order to test our hypothesis, we measured expression levels of LH-β,α subunit and GnRH mRNA using real time RT-PCR, as well as FSH-β mRNA expression using traditional RT-PCR in hippocampal and cortical tissue from AD patients (n=14) and age-matched controls (n=8). We report for the first time to our knowledge the presence of LH-β and GnRH, but not α subunit or FSH-β mRNA in hippocampal and/or cortical tissue in both AD and control brain. This offers support to the notion of an “HPG-like” axis in the brain; however, the lack of α subunit or FSH-β mRNA in the brain suggests that the hormonal axis within the brain is unique and is comprised of different components than the canonical HPG axis. Furthermore, we report a statistically significant increase in LH-β mRNA that is not accompanied by corresponding increases in GnRH mRNA in AD versus control brain. The disparity between LH-β and GnRH mRNA expression levels in AD reinforces the distinctiveness of the brain-specific hormonal axis as increases in LH-β mRNA in the pituitary would likely be accompanied by increases in GnRH mRNA as GnRH governs the synthesis of LH. Finally, increased in LH-β mRNA in the AD brain could potentially account for increased LH protein reported in the hippocampal neurons in AD, and provides a novel therapeutic target for the treatment of AD.

### Materials and Methods

Tissue: Hippocampal or cortical tissue samples were obtained post mortem from patients (n=14, ages 69-96 years) with histopathologically confirmed AD, as well as from aged-matched controls (n=8, ages 71-93 years) with similar post mortem intervals (AD: 5.5-25 h; controls: 6-27 h). All cases were categorized based on clinical and pathological criteria established by CERAD and NIA consensus. From the clinical reports available to us, we found no obvious differences in agonal status or other potential confounders between the groups.

Real-time quantitative PCR analysis: Total RNA was extracted from dissected brain and pituitary and was sent to the Gene Expression and Genotyping Facility at CASE for quantitative analysis of each target gene. A relative qPCR approach was used to quantitate the change in expression of each target gene using TaqMan “assays on demand” made by ABI. ABI reagents and hardware were used throughout. The facility used 1.5 milligrams of total RNA for the reverse transcription step in a 100 ul reaction. The PCR reactions were run on an ABI 7900HT machine using standard manufacturer protocols. PCR assays were executed in triplicate on a 384-well plate with a reaction volume of 15 microliters. A 1/1000 dilution of the RT reaction was used as starting material in the PCR runs.

A preliminary PCR plate run was executed in order to determine a suitable gene for use as an endogenous control for RNA loading. Four candidate genes were assayed. These were: 18S rRNA, GAPDH, β-actin and TATA binding protein. While each of these assays had robust amplification, the β-actin assay displayed the tightest banding across the sample set and was chosen as the endogenous control gene for the main PCR run. A pituitary sample was used as the calibrator sample. All fold changes recorded in the analysis are in reference to this calibrator sample.

RT-PCR: Total RNA was extracted from dissected brain and pituitary using the RNAqueous®4PCR Kit (Ambion, Austin, Tex.). To further reduce the content of genomic DNA, each sample was subjected to DNase treatment prior to PCR using TURBO DNase™ (2 U/μl) (Ambion, Austin, Tex.). After degradation of DNA, total RNA was precipitated with 5M ammonium acetate and linear acrylamide according to kit specifications. Between 1-2 micrograms of each sample was then subjected to reverse transcription using the RETROscript® Kit (Ambion, Austin, Tex.), and the resulting cDNA samples were used in subsequent PCR reactions (Table 2). All primers used in this study were designed to span multiple exons, therefore PCR products resulting from DNA contaminants would be distinctively larger in size compared to the desired cDNA product. Fifteen microliters of each PCR product was electrophoresed on a 2% agarose gel and visualized by ethidium bromide staining. RNA isolated from human pituitary was used as a positive control for all genes included in this study, and all results were confirmed via sequencing.

### Results

In this study, we report for the first time to our knowledge the presence of LH-β and GnRH, but not α subunit or FSH-β mRNA in hippocampal and/or cortical tissue in both AD (n=14) and control brain (n=8) using RT-PCR. Prior to engaging in quantitative real time RT-PCR techniques, we began to investigate the expression of LH-β, FSH-β, α subunit and GnRH mRNA in the hippocampus and cortex using traditional RT-PCR techniques. Pituitary samples were used as positive controls in order to demonstrate the success of each PCR, as all of the hormones investigated in this study are known to be expressed in the pituitary. In concordance with previous studies, we were also able to amplify LH-β, FSH-β, α subunit and GnRH mRNA in the pituitary reflecting the applicability of the RT-PCR techniques used in this study. Furthermore, the housekeeping gene, S15, a small ribosomal subunit, was successfully amplified in all brain and pituitary samples used in this study demonstrating that the RNA isolated from the post-mortem tissue used in this study was of sufficient integrity for RT-PCR.

When both of these positive controls are taken into account, we conclude that unlike LH-β and GnRH, FSH-β and α subunit mRNA were simply not present in either the AD or control hippocampal and cortical tissue samples. Upon completion of our preliminary studies using traditional RT-PCR, we prepared samples for real time RT-PCR analysis in which the relative expression levels of LH-β, α subunit and GnRH mRNA were to be measured. Although in our preliminary studies we did not detect α subunit expression in either the AD or control brain samples, we chose to include this gene in the real time RT-PCR analysis as a means to validate the data obtained from traditional RT-PCR. The results from the real time RT-PCR analysis were consistent with our finding that α subunit mRNA is not expressed in the brain and having successfully validated of our prior RT-PCR experiment, FSH-β was not included in further real time RT-PCR analysis.

| | | | | PCR | |
|---|---|---|---|---|---|
| | | | # of | Product | |
| Primer | Orientation | Sequence | bases | Size | References |
| α subunit | Sense | Gccatggattactacagaaaatat | 24 | 451 bp | (Yokotani et al. 1997 |
| | | (SEQ ID NO: 1) | | | |
| | Antisense | Cagtaaagctgcagtatatccttg | 24 | | |
| | | (SEQ ID NO: 2) | | | |
| LH-β | Outer sense | Ctgttgctgctgctgag | 17 | 404 bp | (Hotakainen et al. |
| | | (SEQ ID NO: 3) | | | 2000) |
| | Outer | Gcctttgaggaagagaggag | 18 | | |
| | antisense | (SEQ ID NO: 4) | | | |
| | Nested sense | Atcctggctgtcgagaagg | 19 | 292 bp | *based on published |
| | | (SEQ ID NO: 5) | | | sequence |
| | Nested | Tggtcacaggtcaaggggtg | 20 | | |
| | antisense | (SEQ ID NO: 6) | | | |
| FSH-β | Sense | Tgttgctggaaagcaatctg | 20 | 202 bp | (Kumar and Low |
| | | (SEQ ID NO: 7) | | | 1995) |
| | Antisense | Cctggctgggtccttataca | 20 | | |
| | | (SEQ ID NO: 8) | | | |
| GnRH | Sense | Tggtgcgtggaaggctgctc | 20 | 228 bp | (Limonta et al. 1993) |
| | | (SEQ ID NO: 9) | | | |
| | Antisense | Cttcttctgcccagtttcctc | 21 | | |
| | | (SEQ ID NO: 10) | | | |

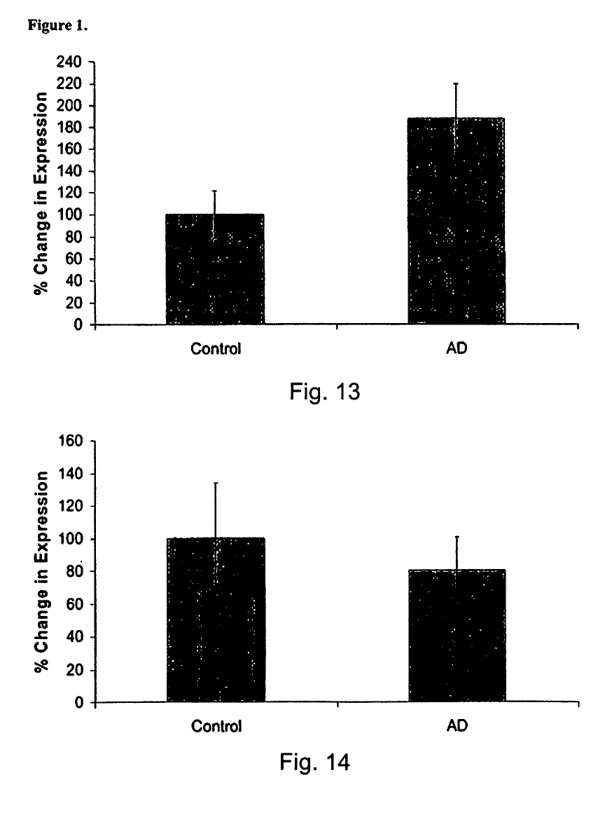

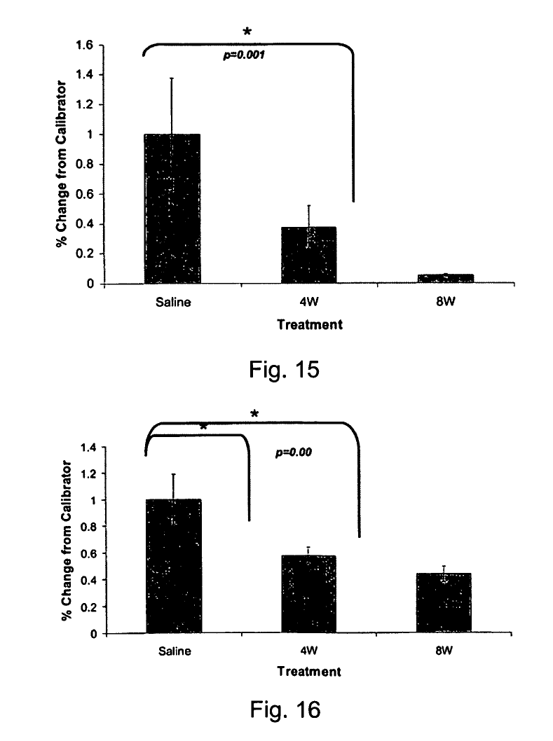

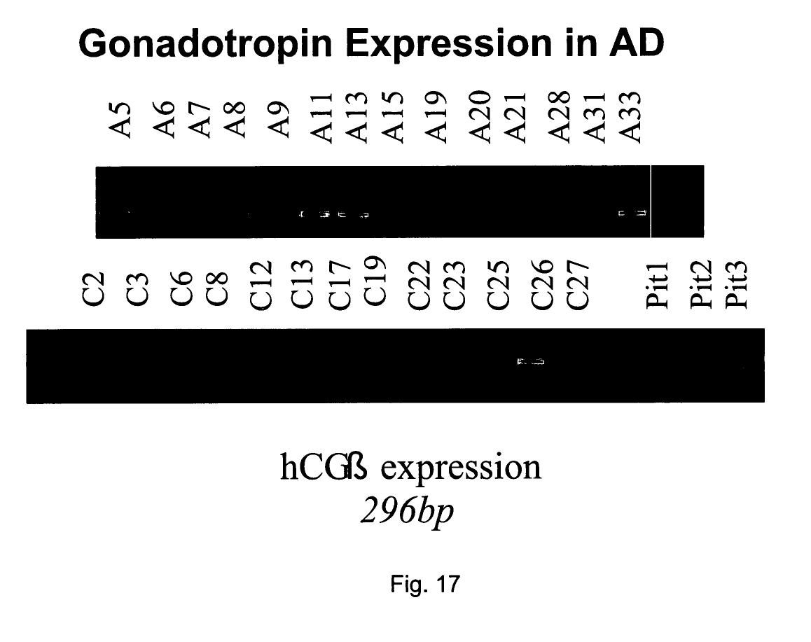

We report a statistically significant increase in LH-β mRNA (FIG. 13) that is not accompanied by corresponding increases in GnRH mRNA (FIG. 14) in AD (n=14) versus control (n=8) brain tissue using real time RT-PCR techniques (p=0.040). As previously mention, α subunit mRNA was not successfully amplified in either the AD or control brain tissues, yet was successfully amplified in the pituitary supporting our previous RT-PCR data. Similarly, LH-β and GnRH mRNA were successfully amplified using real time RT-PCR techniques in hippocampal and cortical tissues from AD and control brain as well as pituitary, confirming data from initial RT-PCR experiments. Increases in LH-β mRNA in AD tended to be more pronounced in AD females compared to AD males although not significantly, and no such trend was observed in GnRH mRNA expression in the AD brain.

### Discussion

In this study, we report for the first time the expression of LH-β mRNA in human cortical and hippocampal brain tissue, and furthermore, we report a statistically significant increase of LH-β mRNA in AD brain tissue in comparison to age-matched, control brain tissue. We also detected GnRH mRNA in cortical and hippocampal tissue; however, there was no difference between GnRH mRNA expression levels in AD versus control tissues. Since LH-β expression is primarily regulated by GnRH, the lack of increased GnRH mRNA in AD is surprising as increases in LH-β mRNA in AD would be expected to be the result of increases in GnRH mRNA in AD. Finally, we report the absence of FSH-β and α subunit mRNA expression in either AD or control cortical and hippocampal tissue suggesting that despite similarities in the promoter regions of LH-β, FSH-β and α subunit genes, there is not only gonadotropin-specific, but also subunit-specific expression in the brain.

In order to simplify our interpretation of these results, we will first discuss the implications of brain-derived gonadotropin subunit expression outside of disease context, as the finding of LH-β and GnRH mRNA in human cortical and hippocampal tissues is, in and of itself, a novel finding. While the endogenous production of gonadotropins by the brain had yet to be studied prior to our investigation, it has been well documented that neurons in various regions of the central nervous system synthesize sex steroids, often termed “neurosteroids” to denote their origin. Neurosteroids are believed to be important for complex neuronal functions including, but not limited to, hippocampal synaptic plasticity. Furthermore, while GnRH mRNA expression has not been reported in hippocampal or cortical tissue aside from the report herein, GnRH mRNA expression has been described in detail in the hypothalamic neurons supporting the notion that neurons in general maybe capable of expressing GnRH, as well as sex steroids, in other regions of the brain. With several components of the HPG axis being synthesized de novo within various regions of the brain, it is not surprising that the brain is also capable of synthesizing gonadotropin subunits as evidenced by the presence of LH-β mRNA in hippocampal and cortical tissue included in this study. Presumably, the potential of gonadotropins to cross the blood-brain barrier has diverted focus away from the study of endogenous, brain-derived gonadotropins, despite evidence in support of such a notion.

The novel finding of LH-β mR.NA in hippocampal and cortical regions of the brain is physiologically consistent with the synthesis of other hormones associated with the HPG axis in the brain, yet the absence of a subunit expression in the brain in this study is more difficult to interpret and prompts further investigation. Like all gonadotropins, LH is a heterodimer consisting of an α subunit that is common to all of the gonadotropins and a β subunit that is gonadotropin-specific. The α and β subunits are non-covalently linked by disulfide bonds and it is thought that the biological activity of LH is dependent upon the formation of the heterodimer. Not surprisingly, the transcriptional regulation of LH-β and α subunit are thought to be coordinately regulated, and furthermore, it is traditionally thought that LH-β secretion is dependent on the presence of a subunit. With this in mind, it is possible that LH-β subunit may not be secreted into the extracellular matrix, and instead has a potentially novel intracellular role in the brain. Considering the fact that LH-β and α subunit do not appear to be coordinately regulated in the brain in this study as is traditionally thought, however, the possibility that LH-β subunit secretion also does not follow the canonical pathway and is secreted into the extracellular matrix as a monomer should be considered. A review of the literature begins to uncover the potential for free LH-β subunit secretion as an earlier study reported that of thirty clones that were positive α/LH-β transformants, fourteen clones were reported to secrete only the LH-β subunit suggesting that LH-β may indeed be able to be secreted as a monomer. It can only be assumed that since the in vitro production of LH dimer was the goal of this experiment, the clones that secreted only LH-13 subunits were not utilized for further study.

While the study mentioned above suggests that free LH-β secretion may be possible, more recent reports of detectable free LH-β in human serum suggests that free LH-β may in fact be physiologically relevant. Normal, postmenopausal women have been shown to have increased basal plasma free LH-β compared to normal men and premenopausal women, and furthermore that free LH-β levels parallel dimeric LH and FAS levels as measured by a very specific and sensitive immunoradiometric assay (IRMA). Furthermore, during the LH surge in women, there is a parallel increase in LH dimer, free LH-β, and FAS in the serum, while free CG-β levels remained undetectable as measured again by a highly specific and sensitive IRMA. Notably, LH, free LH-β and FAS levels were measured in women with functional hypothalamic amenorrhea, who have very low endogenous LH levels and undetectable free LH-β levels, before and after GnRH treatment and recombinant LH treatment. LH, free LH-β, and FAS levels increased in FHA women receiving pulsatile GnRH treatment, and even more importantly, free LH-β and FAS did not increase in an FHA woman upon recombinant LH treatment, suggesting that the free LH-β in the serum is from a pituitary origin and not a product of LH dimer proteolysis. Taken together, these data provide support to the notion that LH-β can be secreted as a free subunit and may not require the presence of the α subunit for secretion and that this monomer may have a physiological role. Although the biological activity of a free LH-β subunit and the potential for its secretion has yet to be determined, further studies are warranted to elucidate the biologic impact of endogenous, brain-derived hormones and their subunits.

As previously mentioned, we report not only the presence of LH-β and GnRH mRNA in the brain, but also a statistically significant increase in LH-β (p<0.05) (FIG. 13), but not GnRH mRNA (FIG. 14) in AD versus age-matched control brain. This suggests a potentially pathogenic role for endogenous, brain-derived LH-β subunit in AD, and furthermore, presents a potential therapeutic target for the treatment of AD. Initially, LH was linked to AD pathogenesis by the report of a two-fold increase in circulating gonadotropins in individuals with AD compared with age-matched control individuals. Furthermore, LH has been shown to alter AβPP processing toward the amyloidogenic pathway, as well as lead to cognitive decline in LH-β transgenic that exhibit elevated LH levels well as increased estradiol and testosterone levels when compared to non-transgenic littermates. Interestingly, significant elevations of LH were not only found in the serum of individuals with AD, but increased LH was also found in vulnerable neuronal populations in individuals with AD compared to aged control. This potentially pathogenic neuronal increase of LH could be the consequence of two different mechanisms, namely that serum LH crossing the blood-brain barrier and subsequently being endocytosed by neurons, or alternatively, that the neurons themselves are synthesizing LH. This seemingly insignificant detail becomes of primary importance when attempting to design a therapeutic strategy such as lowering brain-derived LH, which has different requirements than lowering serum LH due to the blood-brain barrier.

Leuprolide acetate, a potent GnRH agonist that suppresses LH and sex steroid production by down regulating GnRH receptors in the pituitary, became a potential therapeutic and it is currently in phase III clinical trials for the treatment of AD. In support of leuprolide acetate-induced neuroprotection, leuprolide acetate treatment resulted in decreased total brain Aβ1-42 and Aβ1-40 concentrations 3.5-fold and 1.5-fold, respectively, in C57B1/6J mice. Decreases in serum LH levels by leuprolide acetate administration have also been associated with decrease amyloid plaque burden and subsequently increase cognition in AβPP transgenic mice. Despite the evident effectiveness of leuprolide acetate in these studies, the mechanism by which leuprolide acetate promotes neuroprotection in the brain is unclear. Specifically, is the neuroprotection provided by leuprolide acetate the result of decreases in serum LH and therefore the amount of LH that crosses the blood-brain barrier to which the brain is exposed, or the result of direct decreases in endogenously expressed, brain LH. The first scenario requires LH to cross the blood-brain barrier and while the sex steroids are known to cross the blood-brain barrier due to their hydrophobic nature, less is known about the ability of gonadotropins, which are peptide hormones and therefore hydrophilic, to cross the blood-brain barrier. hCG, which is a member of the gonadotropin family, has the ability to cross the blood-brain barrier albeit with low efficiency, but similar studies have not been preformed with LH making it impossible to exclude a potentially direct effect of leuprolide acetate on endogenously expressed LH in the brain. Similarly, because leuprolide acetate has long been thought to solely affect the pituitary, which lies outside the blood-brain barrier, it has yet to be determined if leuprolide acetate is indeed able to gain access to the brain. Notably, GnRH has been shown to cross the blood-brain barrier in a bidirectional, saturable manner, which suggests that leuprolide acetate, a GnRH agonist, may also be able to cross the blood-brain barrier due to structural similarities. In support of this notion, we have recently determined that leuprolide acetate is able to lower brain-derived LH-β mRNA expression in C57B16J mice (unpublished data) and therefore may in fact be the mechanism by which leuprolide acetate offers neuroprotection.

In conclusion, we have demonstrated the presence of LH-β and GnRH mRNA, but not FSHβ or α subunit, in AD and control hippocampal and cortical tissues, and furthermore, we report a statistical increase of LH-β mRNA in AD versus age-matched control brains. To our knowledge, this is the first report of endogenous, brain-derived LH-β expression that is not only gonadotropin-specific, but also subunit specific, and which supports the utilization of hormones by the brain that are traditionally thought to be primarily involved in the endocrine system. Importantly, a pathologic imbalance in these brain-derived hormones is evidenced by a statistically significant increase of LH-β mRNA in AD, uncovering a novel therapeutic target in the treatment of AD.

From the above description of the invention, those skilled in the art will recognize, or be able to ascertain using no more than routine experimentation, numerous equivalents to the compounds and methods of use thereof described herein. Such equivalents are considered to be within the scope of this invention and are covered by the following claims. All patents, publications, and references cited in the present application are herein incorporated by reference in their entirety.

1-8. (canceled)

9. A method of treating or preventing neurodegenerative disease in a subject, the method comprising:
administering to the subject a therapeutically effective amount of at least one physiologically acceptable agent that reduces or eliminates brain-derived gonadotropin and/or gonadotropin receptor levels in the subject.

10. The method of claim 9, the agent reducing or eliminating leutinizing hormone-β levels in the subject.

11. The method of claim 9, the agent being administered to the subject at an amount effective to reduce or eliminate amyloid-β levels in the brain.

12. The method of claim 9, the agent reducing the level of at least one of GnRH or GnRH receptor in the subject's brain.

13. The method of claim 9, the agent comprising at least one of GnRH analogs, GnRH antagonists, GnRH receptor antagonists, anti-GnRH antibody, anti-GnRH receptor antibody, gonadotropin antagonists, gonadotropin receptor antagonists, anti-gonadotropin antibody, or anti-gonadotropin receptor antibody.

14. The method of claim 13, the agent comprising leuprolide or a physically acceptable analogs and salts thereof.

15. The method of claim 13, the agent comprising interference RNA directed to mRNA that encodes gonadotropin, and/or gonadotropin receptor in the brain.

16. A method of treating or preventing Alzheimer disease in a subject, the method comprising:
administering to a subject a therapeutically effective amount of at least one physiologically acceptable agent that reduces or eliminates brain derived gonadotropins and/or brain derived gonadotropin receptors in the subject.

17. The method of claim 16, the brain derived gonadotropin and/or gonadotropin receptor comprising at least one of brain derived luteinizing hormone, brain derived luteinizing hormone receptor, brain derived human chorionic gonadotropin, and brain derived human chorionic gonadotropin receptor.

18. The method of claim 16, the agent reducing or eliminating leutenizing hormone-β levels in the subject.

19. The method of claim 16, the agent being administered to the subject at an amount effective to reduce or eliminate amyloid-β levels in the brain.

20. The method of claim 16, the agent reducing the level of at least one of GnRH or GnRH receptor in the subject's brain.

21. The method of claim 16, the agent comprising at least one of GnRH analogs, GnRH antagonists, GnRH receptor antagonists, anti-GnRH antibody, anti-GnRH receptor antibody, gonadotropin antagonists, gonadotropin receptor antagonists, anti-gonadotropin antibody, or anti-gonadotropin receptor antibody.

22. The method of claim 21, the agent comprising leuprolide or a physically acceptable analogs and salts thereof.

23. The method of claim 21, the agent comprising interference RNA directed to mRNA that encodes gonadotropin, and/or gonadotropin receptor in the brain.
